(19) United States
(12) Patent Application Publication
(10) Pub. No.: US 2017/0203067 A1
(43) Pub. Date: Jul. 20, 2017

(54) Airway and Bite-Block System

(71) Applicant: The Lethean Company LLC, Arundel, ME (US)

(72) Inventors: Peter David Eaton, Arundel, ME (US); Lauren May Eaton, Oakland, CA (US)

(21) Appl. No.: 15/410,766

(22) Filed: Jan. 20, 2017

Related U.S. Application Data
(60) Provisional application No. 62/280,698, filed on Jan. 19, 2016.

(51) Int. Cl. A61M 16/04

(52) U.S. Cl. CPC A61M 16/0497; A61M 16/0493; A61M 13/00

(57) ABSTRACT

An airway system may include an airway and a bite block.

### TECHNICAL FIELD

This disclosure generally relates to oropharyngeal airways and bite blocks.

### BACKGROUND

Patients are commonly anesthetized during minimally-invasive surgeries, including, for example, endoscopic procedures. In an endoscopic procedure, a surgical instrument may be disposed in a patient's upper airway, for example the esophagus or the airway structures distal to the glottic opening, and a protective bite block may be placed in the patient's mouth opening. While the patient is anesthetized, a patent airway must be maintained. As used herein, patent airway may be used to refer to an airway that is unobstructed.

The induction of an anesthetic, for example, intravenous general anesthesia, causes upper airway tone of a patient to relax. This relaxation of the upper airway structures may be severe enough to block the patient's airway (i.e., the airway is not patent), preventing the patient from spontaneously breathing. Relaxation of the patient's upper airway structures may, for example, cause the patient's tongue to relax and to block the flow of air into and out of the lungs (i.e., the airway is not patent).

In order to address this relaxation of a patient's upper airway and to ensure that the patient continues to spontaneously breathe, mechanical rescue maneuvers may be used to restore a patent airway. Mechanical rescue maneuvers may include, as an example, a jaw thrust maneuver and a chin lift maneuver. A chin lift maneuver includes lifting the patient's chin and extending the neck. Mechanical rescue maneuvers may encourage the relaxed tongue to move into a position anterior and superior to the glottic opening, thus allowing the free movement of air during spontaneous ventilation (i.e., establishing a patent airway). Mechanical rescue maneuvers, however, may be unsuccessful in establishing a patent airway. For example, factors including, the depth of the anesthetic employed, the combination of pharmacologic agents administered, and the size of the patient's tongue, or any combination thereof, may affect the success rate of mechanical rescue maneuvers in establishing and maintaining a patent airway, thus allowing the patient to spontaneously breathe. If successful in establishing a patent airway, a mechanical rescue maneuver may still fail to maintain the patent airway (i.e., ensure that the patient's airway remains patent). Should a mechanical rescue maneuver be unsuccessful in establishing and maintaining a patent airway during a procedure (e.g., an endoscopic procedure or other minimally-invasive surgery), the procedure may need to be abandoned, and any surgical instrumentation removed from the oral cavity.

Oral airways may also be used to reestablish a patent airway. U.S. Pat. No. 2,599,521, titled “Respiratory device,” issued Jun. 3, 1952, describes a traditional oral airway device, which may be referred to herein as the Berman Oral Airway. In the event of an obstructed airway during a surgical procedure, the Berman Oral Airway, and other similar oral airway devices, may be inserted into the upper airway of a patient, after all surgical instrumentation has been removed, in order to clear a channel through which the patient may spontaneously breathe. to provide a channel for respiratory purposes, particularly in unconscious patients such as those who have been administered a general anesthetic. It is the purpose of the oral airway to prevent respiratory obstruction by preventing collapse of the pharyngeal tissues and/or obstruction of the pharynx by the tongue.

During minimally invasive surgery, the relaxed tongue of an anesthetized patient may obstruct the movement of air into and out of the glottic opening. When this occurs, the jaw thrust or chin lift rescue maneuvers may be employed to displace the relaxed tongue and to reestablish a patent airway, thereby allowing for spontaneous patient ventilation. In the event that these rescue maneuvers do not provide for the reestablishment and maintenance of a patent airway, the minimally invasive surgery must be abandoned, necessitating the complete removal of the surgical instrument from the oral cavity. Further, in order to reestablish the patent airway, an airway device must be inserted into the oropharyngeal cavity to displace the tongue away from the glottic opening.

### SUMMARY OF PARTICULAR EMBODIMENTS

Particular embodiments may include a bite block. A bite block (i.e., mouth guard or mouth opener) may be used during endoscopic procedures to prevent the patient from biting down on an endoscope or other instrument used during a surgical procedure. During some medical procedures, for example, endoscopic procedures, it is necessary to insert one or more medical instruments, for example, an endoscope, into the mouth and down the trachea or esophagus of a patient. The bite block may protect both the patient's mouth from the one or more instruments and the endoscope from the patient's mouth. The bite block may maintain the patient's mouth in an open position, providing an opening through which a medical instrument can be passed. The bite block may prevent the patient from biting down on the one or more medical instruments used during a procedure.

The bite block may be designed for use with sedation and analgesia delivery and patient-monitoring systems. In order to increase comfort and reduce patient resistance to the advancing of the scope, patients may be sedated during endoscopic procedures. In the case when the particular sedation drugs are respiratory depressants, there exist certain well-known risks related to patient respiration, including hypoventilation, oxygen desaturation, and apnea.

Particular embodiments may include an airway (i.e., a respiratory airway). The airway may be employed in the oropharynx of a semiconscious or unconscious patient and used to allow for uninterrupted, spontaneous or mechanical ventilation by moving the tongue and epiglottis into such a position superior and anterior to the glottic opening. Moving the patient's tongue and epiglottis as described using the airway may provide for the unobstructed flow of air into and out of the lungs. In particular embodiments, the airway may be inserted in the mouth and pharynx of a patient to provide passageways through the mouth through which air may enter and be expelled in breathing when the patient is under the influence of an anesthetic or when natural respiration is rendered difficult by reason of the collapse of the walls of the pharynx or by the obstruction of the pharynx by the tongue sliding back.

In particular embodiments, the bite block and the airway may be used together. The airway and the bite block may be designed to complement each other. The bite block may include a projection (i.e., a boss), and the airway may include an aperture (i.e., a pocket to receive the projection). The projection and aperture may operate as a key and key lock feature. Thus, the airway may be removably attached to the bite block such that the airway is not displaced with respect to the bite block during a surgical procedure while disposed in a patient's upper airway.

### BRIEF DESCRIPTION OF THE DRAWINGS

FIG. 1A illustrates an example elevation view of an airway.

FIG. 1B illustrates an example front view of an airway.

FIG. 1C illustrates an example rear view of an airway.

FIG. 1D illustrates an example top view of an airway.

FIG. 1E illustrates an example cross-sectional view of an airway.

FIG. 1F illustrates an example cross-sectional view of an airway.

FIG. 1G illustrates an example view of a strap retainer of an airway.

FIG. 2A illustrates an example front view of a bite block.

FIG. 2B illustrates an example top view of a bite block.

FIG. 2C illustrates an example elevation view of a bite block.

FIG. 2D illustrates an example cross-sectional view of a bite block.

FIG. 2E illustrates an example cross-sectional view of a bite block.

FIG. 2F illustrates an example view of a retainer of a bite block.

FIG. 3A illustrates an example front view of a bite block with a dental ridge.

FIG. 3B illustrates an example top view of a bite block with a dental ridge.

FIG. 3C illustrates an example elevation view of a bite block with a dental ridge.

FIG. 3D illustrates an example cross-sectional view of a bite block with a dental ridge.

FIG. 3E illustrates an example cross-sectional view of a bite block with a dental ridge.

FIG. 3F illustrates an example view of a retainer of a bite block with a dental ridge.

### DESCRIPTION OF EXAMPLE EMBODIMENTS

In particular embodiments, an airway system may include an oral airway and a bite block. The airway and the bite block may be used together as a system. The airway may be designed for removable insertion straightforward into the bite block.

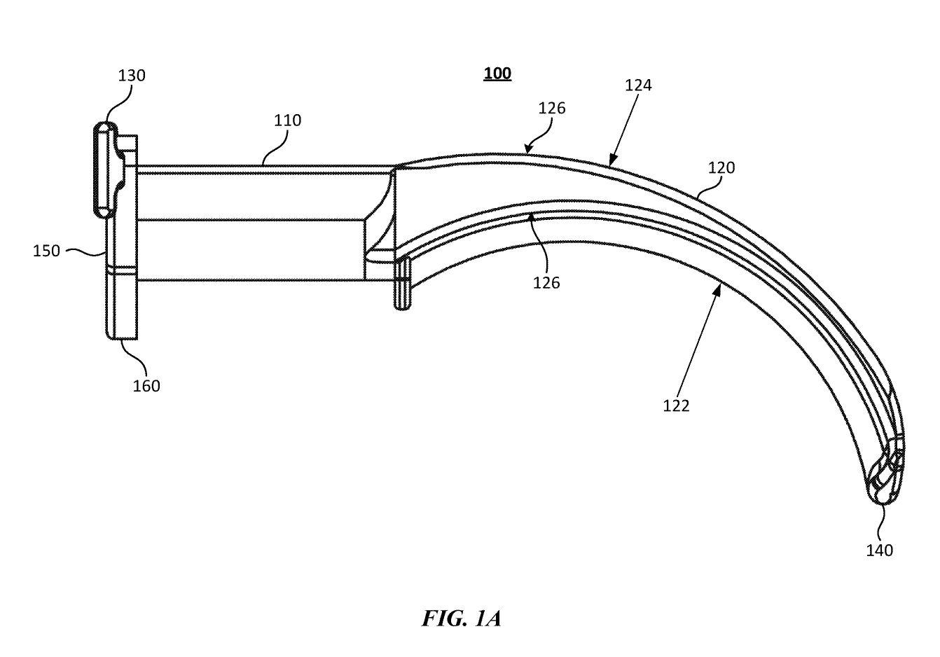

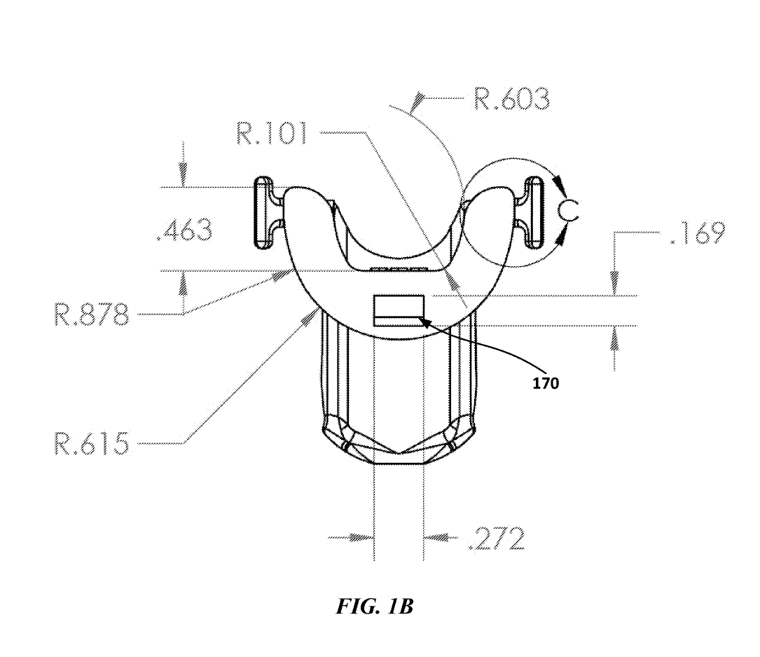

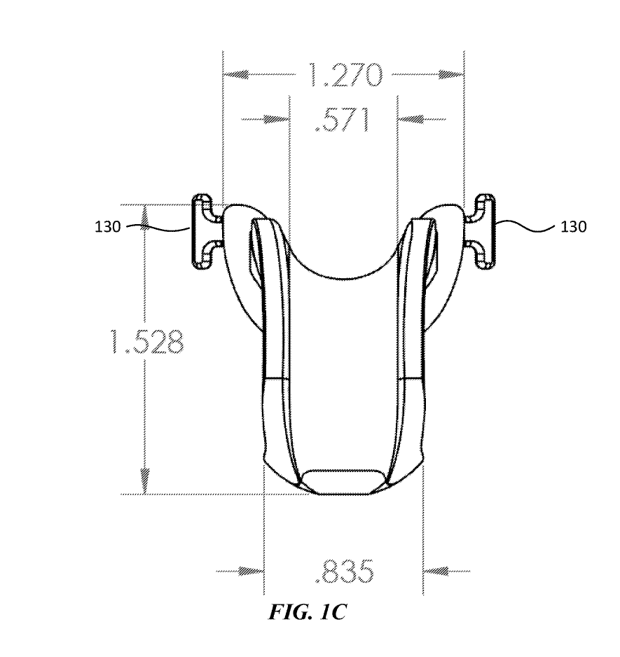

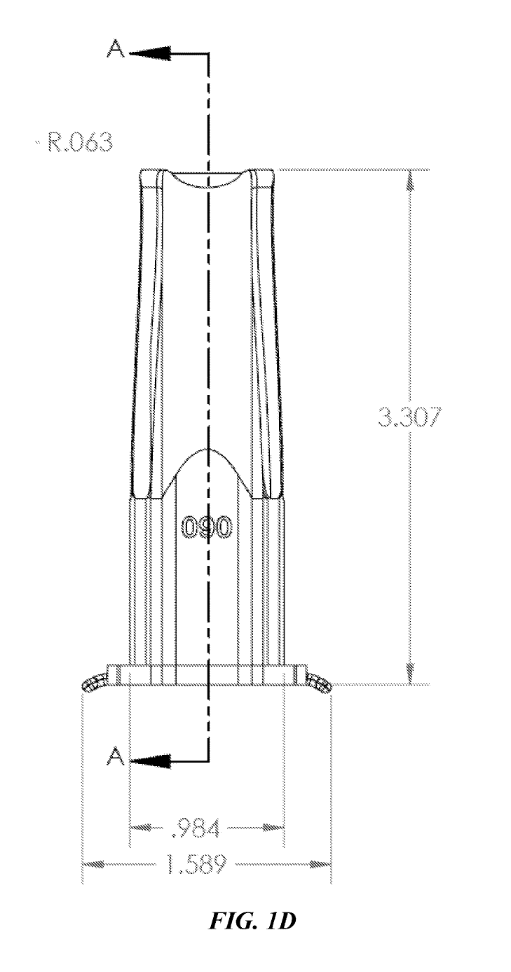

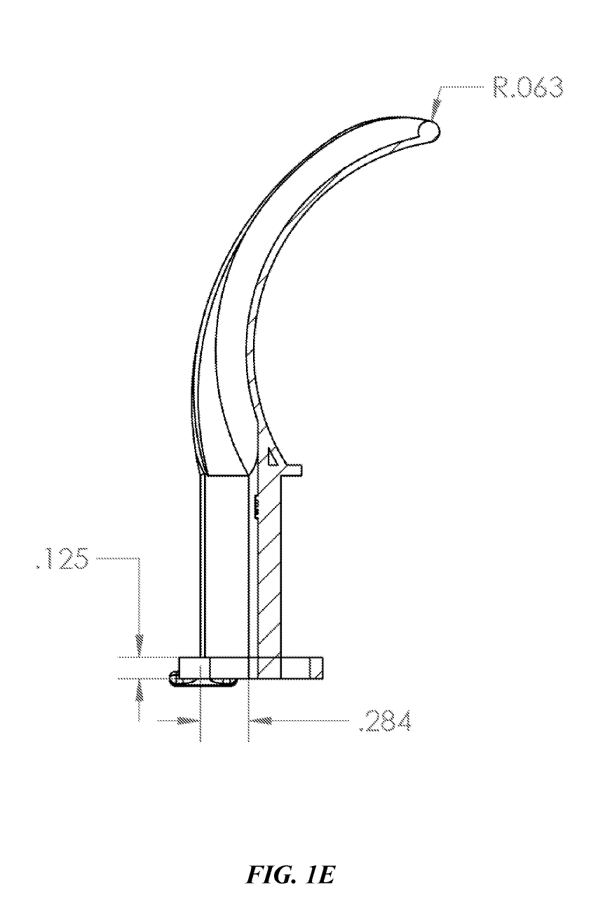

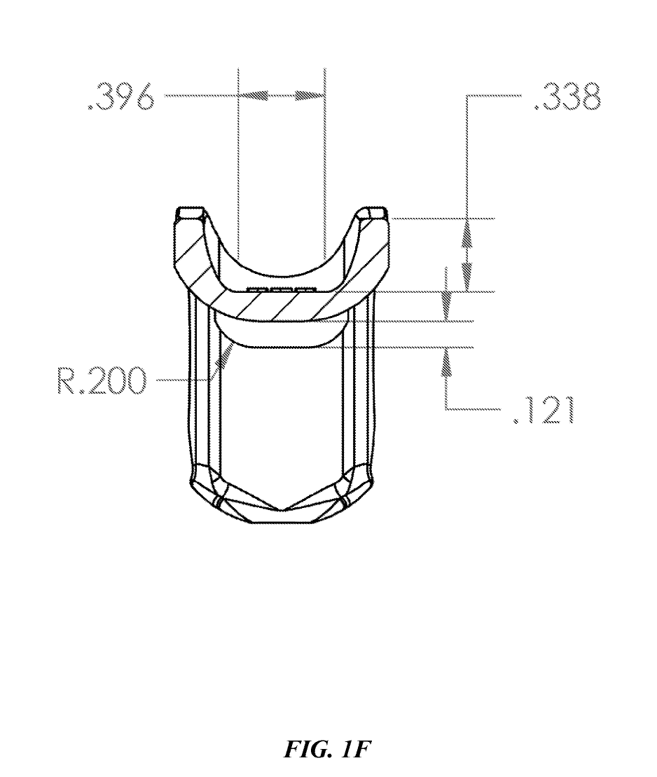

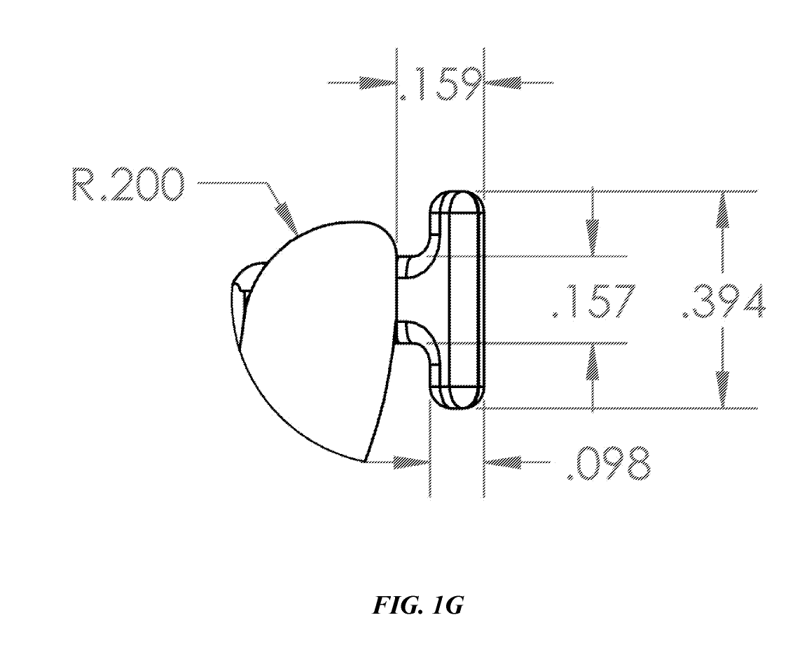

FIGS. 1A-1G illustrate example views of airway 100. FIG. 1A illustrates an example elevation view of airway 100. FIG. 1B illustrates an example front view of airway 100. FIG. 1C illustrates an example rear view of airway 100. FIG. 1D illustrates an example top view of airway 100. FIG. 1E illustrates an example cross-sectional view of airway 100. FIG. 1F illustrates an example cross-sectional view of airway 100. FIG. 1G illustrates an example view of a strap retainer of airway 100.

Airway 100 may include proximal end 150 and distal end 140. It will be understood that airway 100 may be of any suitable size and dimensions. For example, airway 100 may be any suitable length. As an example, airway 100 may be between 3 inches and four inches long from proximal end 150 to distal end 140. Airway 100 may include an upper portion 110 and a lower portion 120. In particular embodiments, the length of upper portion 110 may be greater than the length of lower portion 120. For example, upper portion 110 may be approximately one inch long, and lower portion 120 may approximately two inches long. In another example, upper portion 110 Upper portion 110 may be substantially straight. Lower portion 120 may be a radially-arched channel with a bottom radius 122 and a top radius 124. As an example, lower portion 210 may be a radially-arched channel with a bottom radius 122 of approximately 1.25 inches and a top radius 124 of approximately 1.875 inches. As another example, lower portion 210 may be a radially-arched channel with a bottom radius 122 of approximately 1.215 inches and a top radius 124 of approximately 1.875 inches. As another example, lower portion 210 may be a radially-arched channel with a bottom radius 122 between 1.2 and 1.5 inches and a top radius 124 of between 1.8 and 2 inches. In particular embodiments, lower portion 120 of airway 100 may have a cross section that is substantially U-shaped.

In particular embodiments, airway 100 may include two upstanding walls 126 that taper down to a midpoint on the radius of the airway to prevent injury to the hard palate (e.g., when inserted into a patient's mouth). In particular embodiments, upstanding walls 126 may be symmetric. In particular embodiments, the height of each of the upstanding walls 126 may taper longitudinally down a length of lower portion 120. In particular embodiments, the taper of upstanding walls 126 may be formed by radius 122 and radius 124. In particular embodiments, distal end 140 may taper to prevent injury to a patient's upper airway structures.

In particular embodiments, airway 100 may have two strap retainers 130. Strap retainers 130 may be attached to the outer edges of the proximal end 150 of airway 100. Strap retainers 130 may be connected to an elastic neck strap that may prevent the accidental displacement of airway 100 from a bite block. Strap retainers 130 may be substantially T-shaped.

In particular embodiments, airway 100 may have a flange 160 at proximal end 150. Flange 160 may be transversely opposed with respect to airway 100. As shown in FIG. 1B, in particular embodiments, airway 100 may have an aperture 170, which is described in further detail below.

Airway 100 may be any suitable size. For example airway 100 may be a 100-millimeter size or a 90-millimeter size. An airway 100 in the 100-millimeter size may have a larger radius 122 than the 90-millimeter airway model. Thus, lower portion 120 of an 100-millimeter size airway 100 may rise to a higher point in the oropharynx than the lower portion 120 in the 90-millimeter size airway 100 and be in closer proximity with the patient's hard palate when inserted into the patient's mouth. In particular embodiments, the airway may have one or more of the features of the airway described in co-pending U.S. patent application Ser. No. 13/543,739, filed 6 Jul. 2012, which is incorporated by reference herein.

Airway 100 may be designed for removable insertion into a bite block using the straightforward technique or with the inverted then rotated 180-degree method.

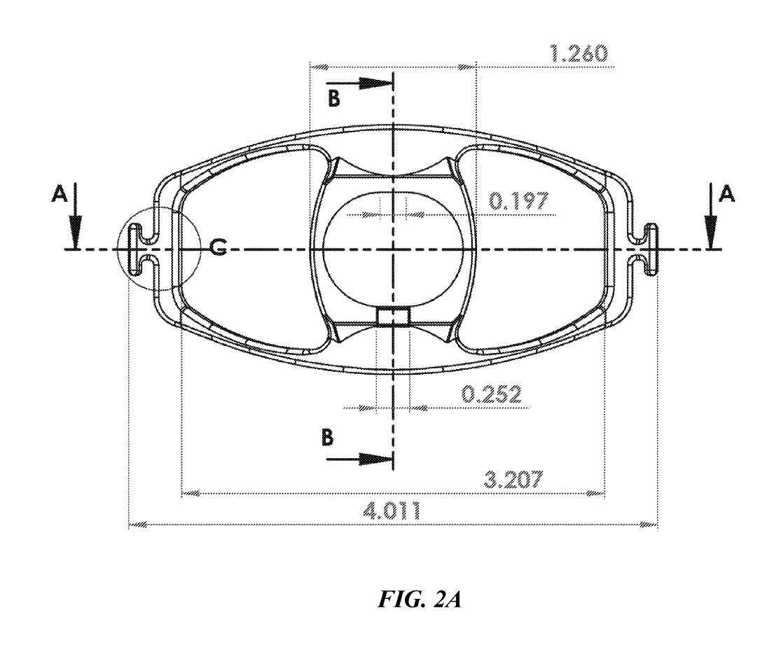

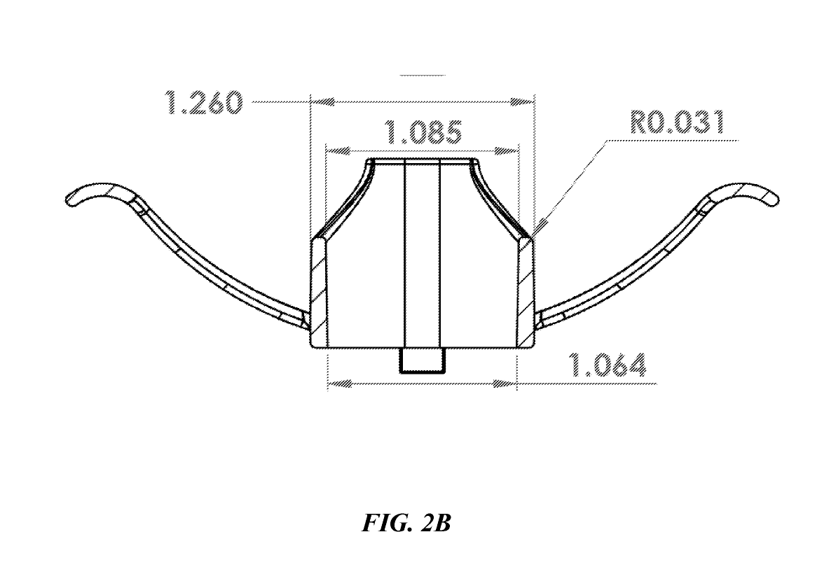

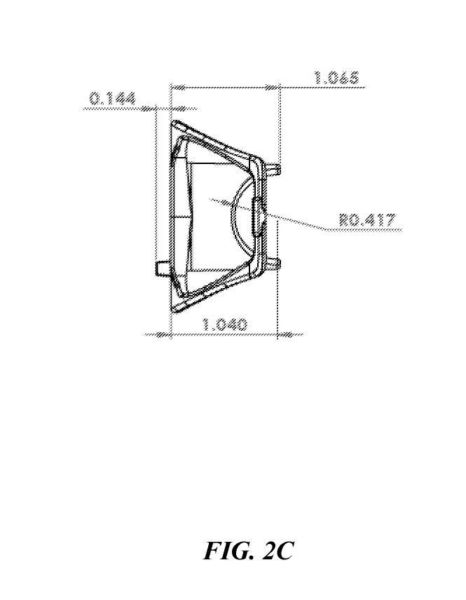

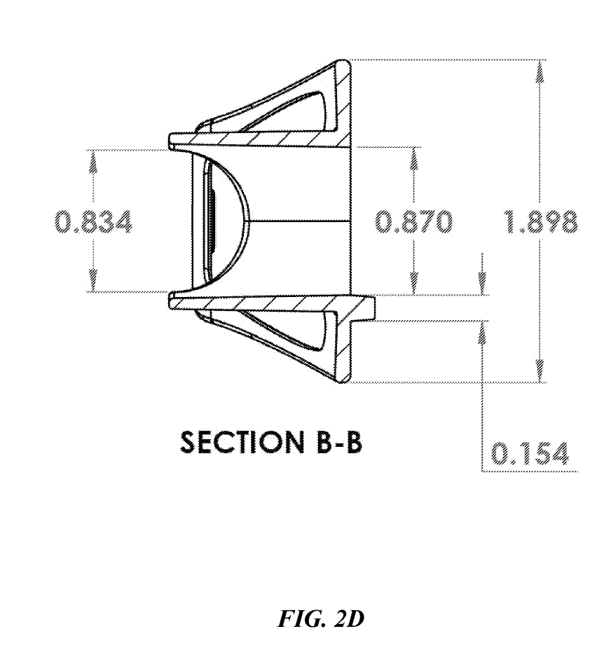

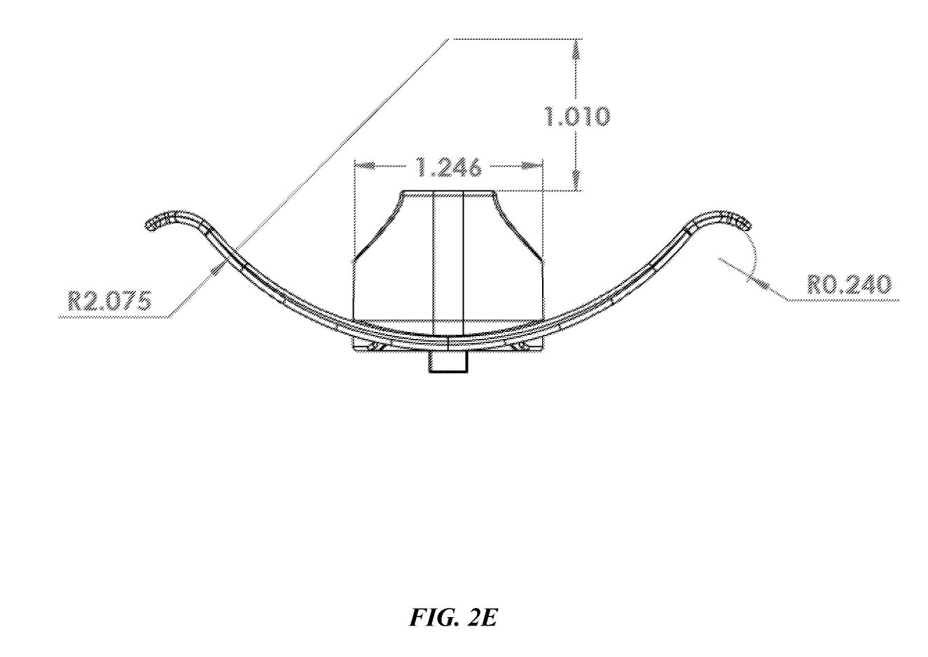

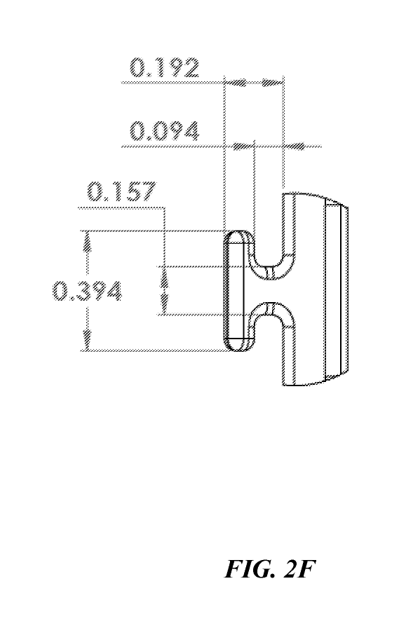

FIGS. 2A-2F illustrate example views of a bite block 200. FIG. 2A illustrates an example front view of a bite block 200. FIG. 2B illustrates an example top view of a bite block 200. FIG. 2C illustrates an example elevation view of a bite block 200. FIG. 2D illustrates an example cross-sectional view of a bite block 200. FIG. 2E illustrates an example cross-sectional view of a bite block 200. FIG. 2F illustrates an example view of a retainer of a bite block 200.

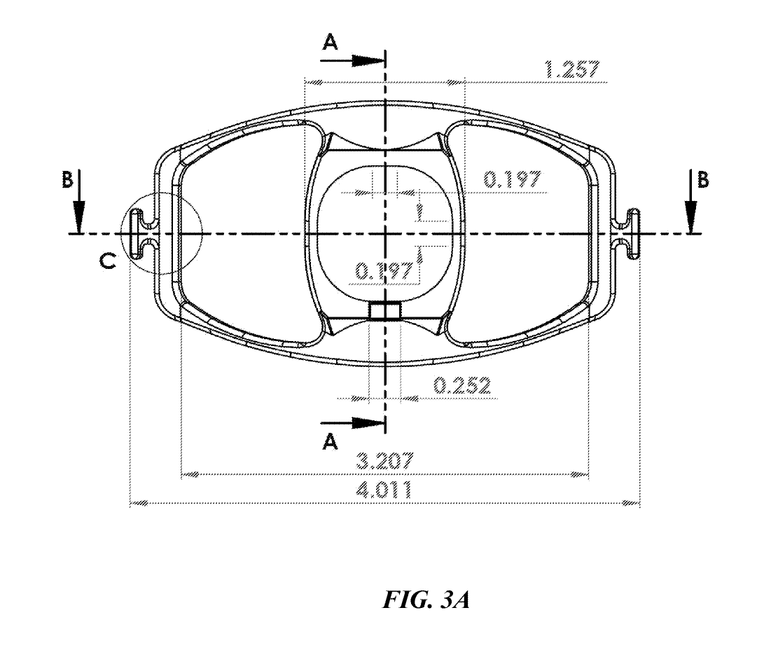

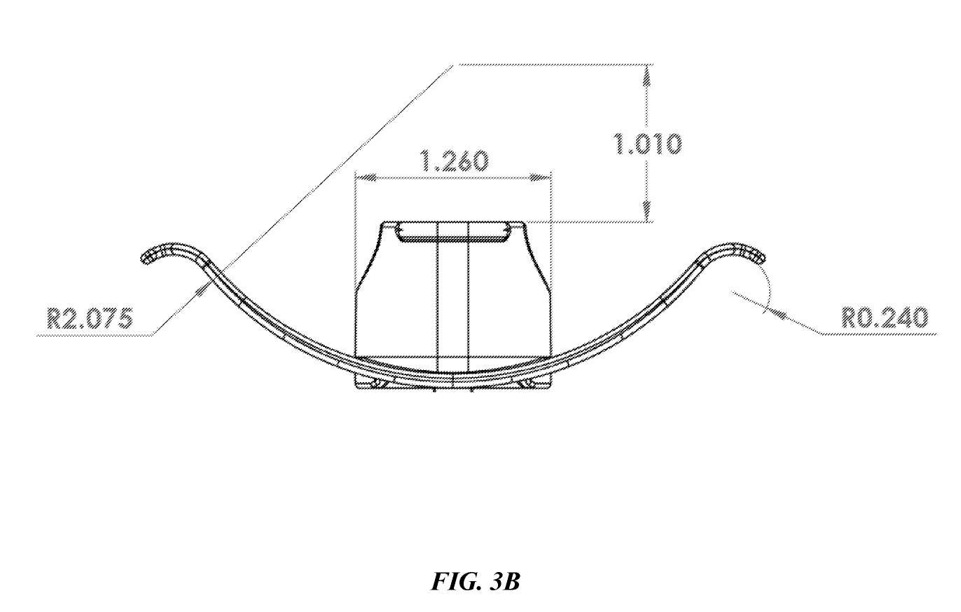

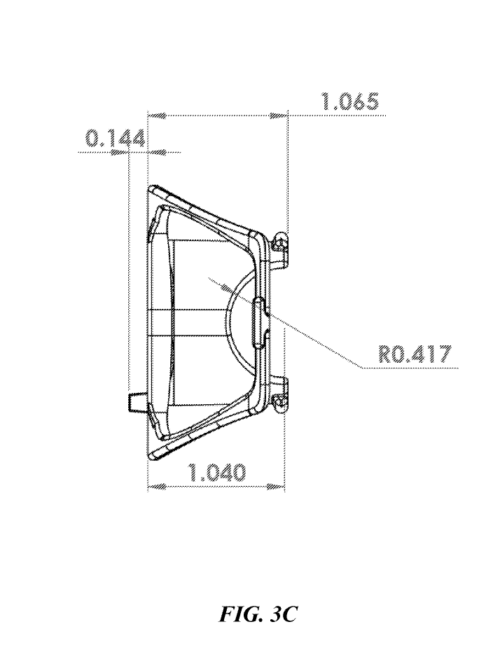

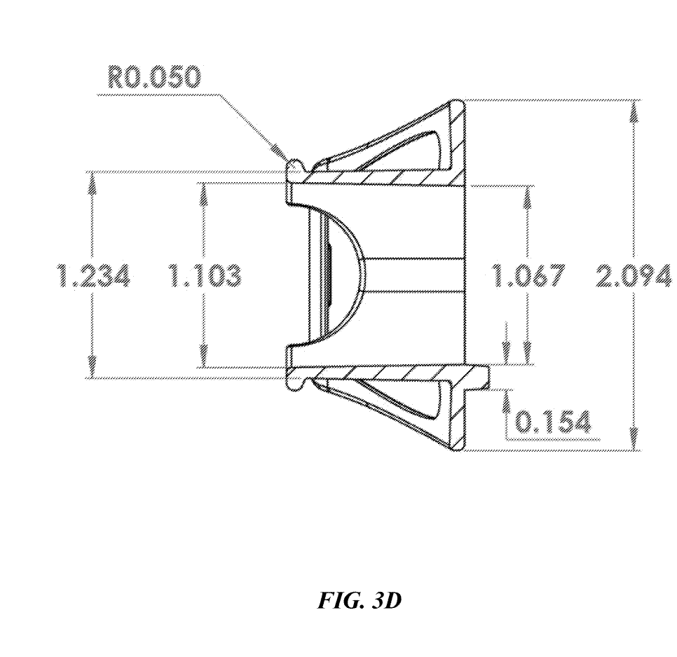

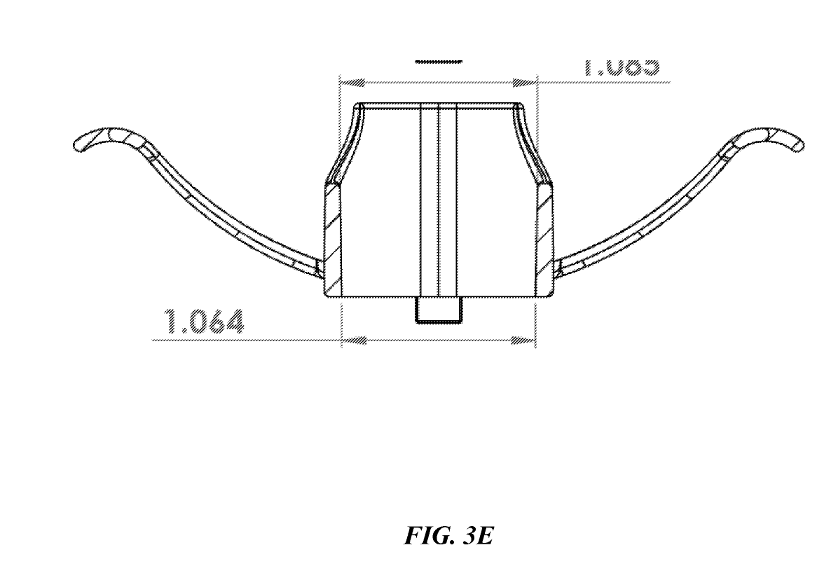

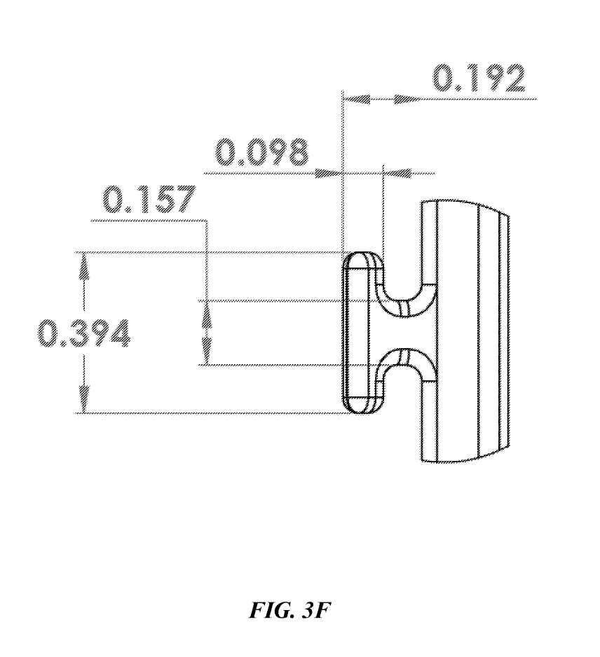

FIGS. 3A-3F illustrate example views of a bite block 200 with dental ridge 300. FIG. 3A illustrates an example front view of a bite block 200 with a dental ridge. FIG. 3B illustrates an example top view of a bite block 200 200 with a dental ridge 300. FIG. 3C illustrates an example elevation view of a bite block 200 200 with a dental ridge 300. FIG. 3D illustrates an example cross-sectional view of a bite block 200 200 with a dental ridge 300. FIG. 3E illustrates an example cross-sectional view of a bite block 200 with a dental ridge 300. FIG. 3F illustrates an example view of a retainer of a bite block 200 with a dental ridge 300. Bite block 200 depicted in FIGS. 3A-3F may be the same as bite block 200 shown in FIGS. 2A-2F, with the exception of dental ridge 200. Dental ridge 200 may be used to maintain the position of the bite block against a patient's dentition. A bite block 200 without a dental ridge 300 may be used with patients who have little or no bottom dentition.

Bite block 200 may have an access hole 210.

The bite block may be available in any suitable size. An 18-millimeter model and a 23-millimeter model may be example sizes of the bite block. An example 18-millimeter bite-block model is depicted in FIGS. 3A-3F. An example 23-millimeter bite-block model is depicted in FIGS. 4A-4F. The 18 mm bite block is a smaller version and may be comfortably used with people with limited mouth opening (i.e., compatible with the 18-millimeter size). The 23 mm bite block is a larger version and may be used with people with a larger mouth opening (i.e., compatible with the 23-millimeter size). The 23-millimeter version may be large enough to allow for the passage of, for example, a 60 French rubber dilator while an airway in place in the bite block.

In particular embodiments, each bite block size may be compatible with each airway size. For example, an 18-millimeter bite block may be employed with either the 90-millimeter or 100-millimeter size airways 100. Although particular sizes of bite blocks 200 are described herein, it will be understood that airway 100, bite block 200, or both may be made in different sizes for use with patients of all ages and dimensions.

In particular embodiments, the airway and bite-block system may include a locking mechanism. In particular embodiments, the locking mechanism may be a key and keyhole type interface. In particular embodiments, the locking mechanism may include a projection (i.e., a boss) and an aperture 170. For example, a bite block may include a projection positioned on a face of the bite block 200 (i.e., a key), and the airway may include an aperture 170 positioned on an outer flange of the airway (i.e., a keyhole). The projection may be, for example, a boss or protruding feature, of any suitable shape and any suitable size. The projection (i.e., boss) may be positioned on a face of a bite block 200. The aperture 170 may be, for example, a pocket or hole on a flange of the airway that is of any suitable shape and any suitable size. The projection and the aperture may be of complementary sizes and shapes such that the aperture may receive the projection. For example, the aperture may be a mating part that receives the projection. As another example, the projection on a face of the bite block 200 (i.e., key) may be have a substantially square shape, rectangular shape, circular shape, or any other suitable shape. As another example, the projection and apperature 170 (i.e., key and keyhole) interface may be of any suitable shape or size. In particular embodiments, when the airway and the bite block are connected such that the projection on the bite block fits into the aperture on the airway, the locking mechanism (i.e., combination projection and aperture) may prevents the rotation of the airway with respect to the bite block. In particular embodiments, when the airway and the bite block are connected such that the projection on the bite block fits into the aperture on the airway, the locking mechanism prevents upward movement of the airway with reference to the bite block.

The portions of the airway 100 that interface with the bite block when used as a system may be designed to be compatible with and to securely interface with bite block 200. For example, the airway may have side walls/upstanding walls having a length and radius designed to be compatible with the bite block. As another example, a stop flange on the distal airway interface may prevent accidental displacement from bite block. The outer surface of the bite-block flange may be a flattened plane that allows for the full contact of the inner surface of the airway flange preventing incidental movement.

The bite block may have retainer supports 220 that may be located sufficiently far apart (with respect to each other) to allow for a large access window to both sides of the bite-block center orifice. Should the user (e.g., a user performing an endoscopy) guide the insertion of an instrument (e.g., an endoscope or an airway) over the surface of the tongue, the user may safely insert his or her finger through the access window of the bite block, and the bite block may prevent the patient from biting down on the user's finger.

In particular embodiments, the airway, the bite block, or both may be constructed of any suitable material, including, for example, metal or plastic. In particular embodiments, the airway, the bite block, or both may be constructed of any suitable recyclable or biodegradable material, including, for example, a starch-based biodegradable polymer. In particular embodiments, the airway, the bite block, or both may be injection molded.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

1. An oral airway system, the system comprising:
an elongated tubular member, wherein the elongated tubular member is removably attached to the airway;
an oral airway for insertion into the oropharyngeal cavity of a patient, the oral airway comprising:
an upper portion and a lower portion, wherein:
the lower portion comprises a radially-arched channel,
the lower portion has a substantially U-shaped cross-section,
the upper portion comprises an aperture.

2. The oral airway system of claim 1, wherein the airway is between three and four inches in length, from proximal end of the airway to distal end of the airway.

3. The method of claim 1, wherein upper portion is substantially straight.

4. The oropharyngeal airway of claim 1, further comprising a flange, wherein the flange is disposed at the proximal end, wherein the flange abuts the protective bite block, the proximal end being prevented from moving through the access hole and from entering the oropharyngeal cavity of the patient by the flange.

5. The airway system of claim 1, wherein the oral airway is comprised of a recyclable material.

6. The airway system of claim 1, wherein the oral airway is comprised of a biodegradable material.
